(19) United States
(12) Patent Application Publication
(10) Pub. No.: US 2014/0024853 A1
(43) Pub. Date: Jan. 23, 2014

(54) Synthesis of Lactic Acid and Alkyl Lactate from Carbohydrate-Containing Materials

(71) Applicants: Xiaoping Zhou, Huzhou City (CN); Jiaruo Huang, Huzhou City (CN)

(72) Inventors: Xiaoping Zhou, Huzhou City (CN); Jiaruo Huang, Huzhou City (CN)

(73) Assignee: MICROVAST, INC., Stafford, TX (US)

(21) Appl. No.: 13/884,607

(22) PCT Filed: Nov. 10, 2011
(86) PCT No.: PCT/US2011/060268

§ 371 Date: May 10, 2013

Related U.S. Application Data
(60) Provisional application No. 61/412,042, filed on Nov. 10, 2010.
(60) Provisional application No. 61/495,431, filed on Jun. 10, 2011.

(51) Int. Cl. C07C 67/40; C07C 51/16

(52) U.S. Cl. CPC C07C 67/40; C07C 51/16
USPC 560/179

(57) ABSTRACT

A method for synthesizing lactic acid and lactate is invented from carbohydrates, such as monosaccharides and/or polysaccharides in the presence of the catalyst that is the combinations of nitrogen-heterocycle aromatic ring cation salts and metal compounds. In the reaction, at least one alcohol and at least one solvent are used. Specifically, in the presence of [SnCl₄-1-ethyl-3-methylimidazolium chloride ([EMIM]Cl)], SnCl₄-1,3-dimethylimidazolium methyl sulfate ([DMIM]CH₃SO₄)], [SnCl₂-1-ethyl-3-methylimidazolium chloride ([EMIM]Cl)], or SnCl₂-1,3-dimethylimidazolium methyl sulfate ([DMIM]CH₃SO₄)] in methanol.

### CROSS REFERENCE TO RELATED APPLICATIONS

This application claims priority to U.S. Provisional Application Nos. 61/412,042, filed Nov. 10, 2010 and 61/495,431, filed Jun. 10, 2011.

### FEDERALLY SPONSORED RESEARCH STATEMENT

Not applicable.

### FIELD OF THE INVENTION

This invention relates generally to a method for synthesizing lactic acid and alkyl lactate from the direct conversion of carbohydrate-containing raw materials, such as monosaccharides and/or polysaccharides, over catalysts in solvent.

### BACKGROUND OF THE INVENTION

Glucose, sugarcane, starch, and celluloses are the most abundant renewable carbon sources found naturally on earth. The high content of oxygenated functional groups in these carbohydrates has advantages in making use of them to produce fundamental chemicals. In particular, these carbohydrates are the most attractive feedstocks for intermediate chemical production in a sustainable way without emitting CO₂.

Theoretically, two moles of lactic acid could be obtained from one mole of hexose either from fermentation or from catalytic reaction. Lactic acid itself is a monomer for the biodegradable polylactate synthesis. Lactic acid and its derivatives (alkyl lactates and polylactate) could act as platform compounds for the synthesis of other carbon-3 building blocks, such as propylene glycol, acrylic acid, and allyl alcohol for the productions of polymers.

Lactic acid is produced from the fermentation of glucose in present chemical industry. In the fermentation process, only very diluted lactic acid broth (<10% water solution) is obtained through reacting with Ca(OH)₂ to obtain calcium lactate solid, and then reacting with a H₂SO₄ solution to isolate lactic acid. The fermentation process generates huge amounts of waste water and CaSO₄ solid waste. The fermentation process for lactic acid production only uses glucose as the feed stock. During production, if starch is used as the feed stock, the starch must be prehydrolyzed to glucose either by acid catalyzed chemical reaction, or by fermentation. Existing fermentation processes could produce lactic acid from glucose in large scale (120,000 tons/year). However, the biological processes generally suffer from low reaction rates and low product concentration (in water), resulting in long reaction times, larger reactors, and high energy consumption in the product purification process (Fermentation of Glucose to Lactic Acid Coupled with Reactive Extraction: Kailas L. Wasewar, Archis A. Yawalkar, Jacob A. Moulijn and Vishwas G. Pangarkar, Ind. Eng. Chem. Res. 2004, 43, 5969-5982). It is known that, in the presence of aqueous alkali hydroxides, monosaccharides can be converted to lactate (R. Montgomery, Ind. Eng. Chem, 1953, 45, 1144; B. Y. Yang and R. Montgomery, Carbohydr. Res. 1996, 280, 47). However, the stoichiometric amount of base (Ca(OH)₂) and acid (H₂SO₄) in the lactic acid recovery process would be consumed and, therefore, the stoichiometric amount of salt waste would be produced. Although the commercial fermentation approach can produce large scale lactic acid, it only uses starch as a feedstock and the starch must be prehydrolyzed (or through fermentation) to glucose in advance. The fermentation process produces large amounts of waste water and solid waste (CaSO₄). The fermentation process for producing lactic acid includes many steps which consume substantial amounts of energy. The infrastructure of the fermentation process is very complicated and uneconomical. FIG. 1 is the scheme of the commercial fermentation process for the production of lactic acid and its derivatives.

It is desired to have a process to convert both monosaccharides and/or polysaccharides to lactic acid and its derivatives directly in a more efficient and economical way. The current invention provides a method for converting monosaccharides and/or polysaccharides to lactic acid and lactate over a homogeneous catalyst system. The catalysts are combinations of nitrogen-heterocycle aromatic ring cation salts and metal compounds dissolved in a solvent. Presently, very few compounds of commercial interest are directly obtainable from carbohydrates by using non-fermentation approaches. There is also no other approach available for the production of lactic acid and its derivatives directly from naturally occurring carbohydrates, such as sugarcane, starch, and cellulose.

### SUMMARY OF THE INVENTION

The present invention provides a method for synthesizing lactic acid and alkyl lactate, comprising: (a) preparing a mixture of at least one carbohydrate-containing raw material, at least one alcohol, at least one catalyst comprised of nitrogen-heterocycle aromatic cation salts and metal compounds, and at least one solvent; and (b) heating the mixture to obtain lactic acid and alkyl lactate.

In addition, polylactic acid can be obtained in the resultant mixture in step (b).

The alkyl lactate in the current invention is selected from the group consisting of methyl lactate and ethyl lactate.

The carbohydrate is selected from the group consisting of polysaccharides and monosaccharides. More specifically, the carbohydrate is selected from the group consisting of cotton, cellulose, starch, dextran, sucrose, fructose and glucose. All substances, which could be converted into carbohydrates by fermentation, hydrolysis, or alcoholysis, can be employed as the reactants of the current invention.

The alcohol is selected from the group consisting of monohydroxyl alcohols, dihydroxyl alcohols, and multihydroxyl alcohols. Further, the monohydroxyl alcohol is selected from the group consisting of methanol, ethanol, 1-propanol, 2-propanol, 1-butanol, 2-butanol, and tert-butanol. The dihydroxyl alcohol is selected from the group consisting of ethylene glycol, 1,2-propandiol, and 1,3-propandiol. The multihydroxyl alcohol is glycerol.

The nitrogen-heterocycle aromatic cation salts in the catalyst of the current invention are comprised of cations and anions. The anion of the nitrogen-heterocycle aromatic cation salts is selected from the group consisting of F⁻, Cl⁻, Br⁻, I⁻, CH₃SO₄⁻, CH₃SO₃⁻, C₆H₅SO₃⁻ (benzenesulfenate anion), SO₄²⁻, HSO₄⁻, H₂PO₄⁻, HPO₄²⁻, PO₄³⁻, PF₆⁻, BO₂⁻, BF₄⁻, SiF₆²⁻, and CH₃CO₂⁻—.

The cation of the nitrogen-heterocycle aromatic cation salts is an organic cation that contains at least one hex-member aromatic ring and/or at least one pent-member aromatic ring that contains at least one nitrogen atom on the ring and carries a positive charge.

More specifically, the cation is an organic cation that contains a hex-member aromatic ring and/or a pent-member aromatic ring that contains at least one nitrogen atom on the ring and carries a positive charge. Yet more specifically, the organic cation is selected from the group consisting of:

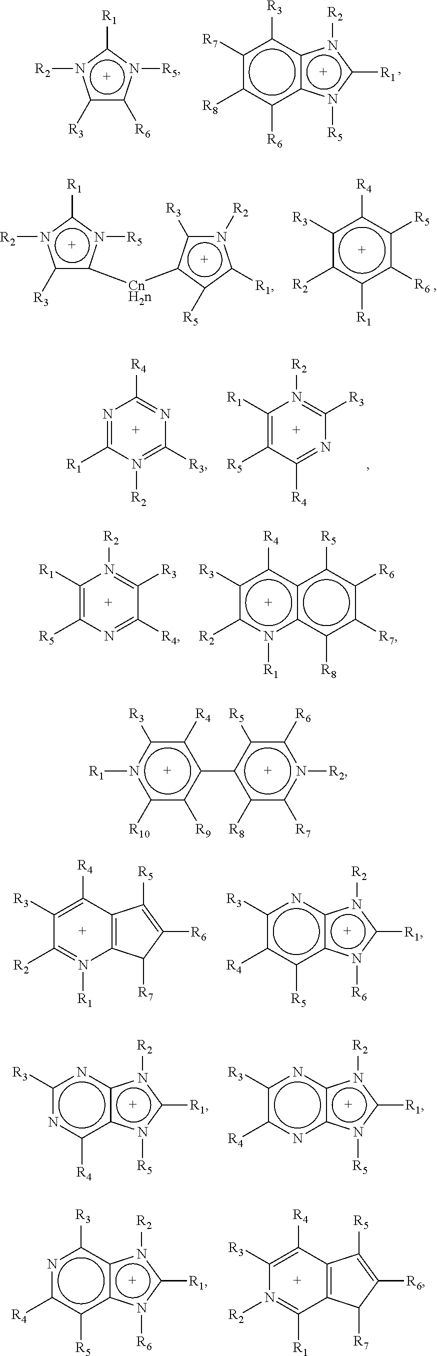

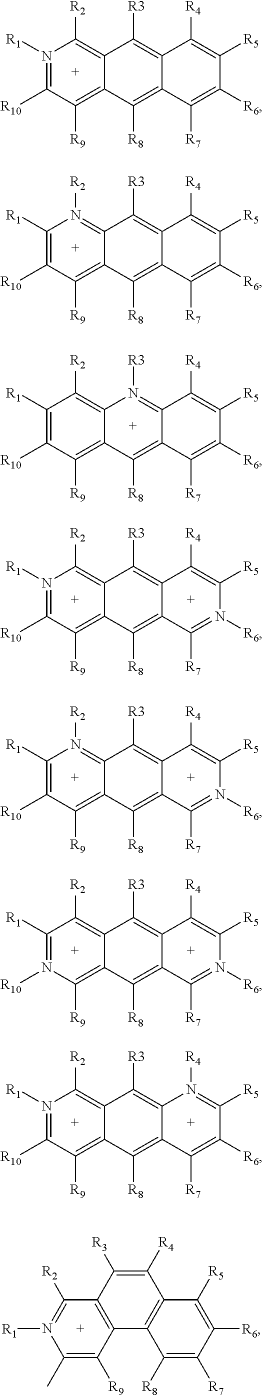

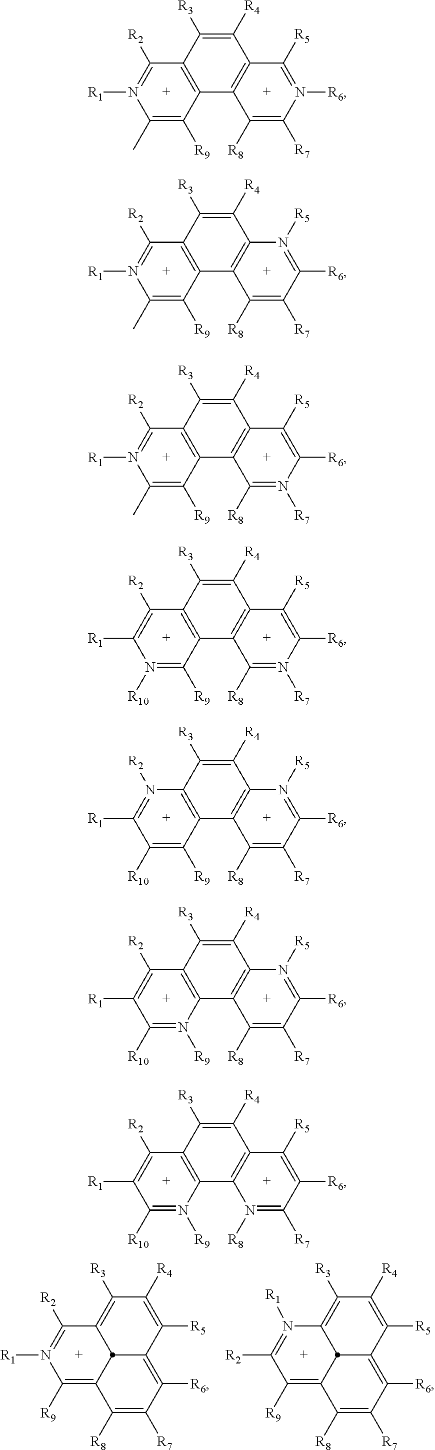

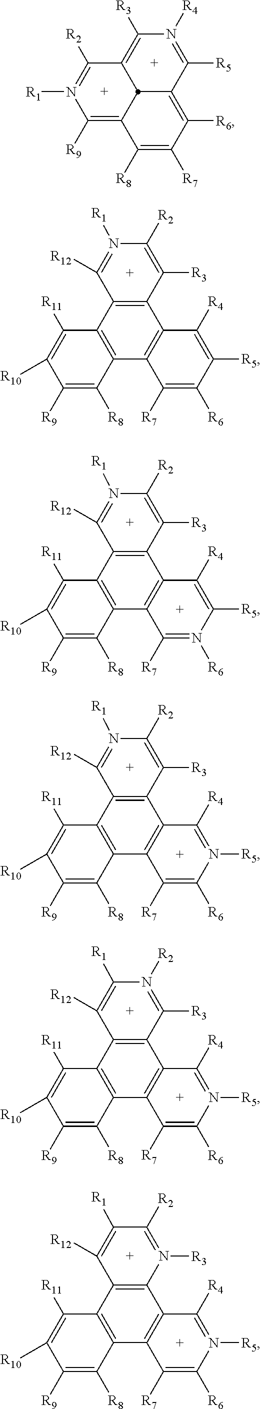

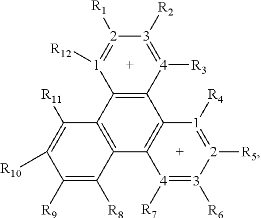

(wherein the two Nitrogen atoms could be on 1, 2, 3, and 4 positions for each ring per N),

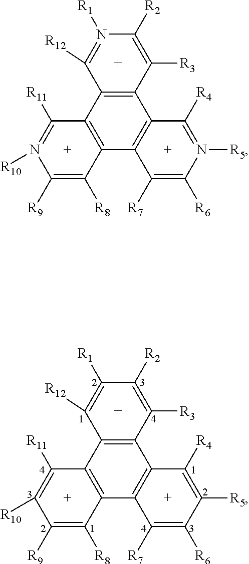

(wherein the three Nitrogen atoms could be on 1, 2, 3 and 4 positions for each ring per N atom),

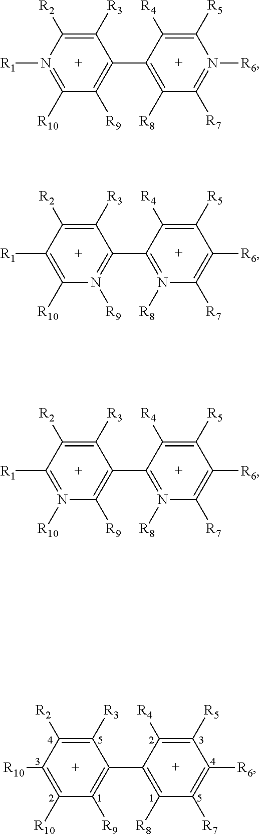

(wherein the two Nitrogen atoms on the two hex-member rings (each ring per N atom) could take any position among 1, 2, 3 and 4),

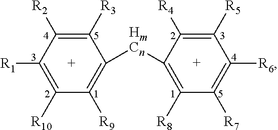

(wherein the two Nitrogen atoms on the two hex-member rings (each ring per N atom) could take any position among 1, 2, 3, and 4; n and m are positive integers), and derivatives thereof; the substituting group Rₙ on carbon atoms is selected from the group consisting of H—, CₙH₂ₙ₊₁— (n≧1), CₙH₂ₙ₋₁—, CₙH₂ₙ₋₃—, CₙHₘ—(m≧3), CₙH₂ₙ₋₇— (n≧6), Cl—, Br—, I—, and —OSO₃⁻. The substituting group Rₙ on nitrogen atoms is selected from the group consisting of CₙH₂ₙ₊₁— (n≧1), CₙH₂ₙ₋₁—, CₙH₂ₙ₋₃—, CₙHₘ— (m≧3), and CₙH₂ₙ₋₇— (n≧6).

In a specific embodiment, the organic cation is selected from the group consisting of 1,3-dimethylimidazolium, 1-ethyl-3-methylimidazolium ([EMIM]⁺), and 1,3-dimethylimidazolium ([DMIM]⁺)

The metal compound in the catalyst of the current invention is selected from the group consisting of Sn, Ti, Zr, and Ge. A useful metal compound for the conversion of carbohydrate-containing raw material is preferably a tin-containing compound, wherein the tin-containing compound comprises Sn⁴⁺, Sn²⁺, or mixtures thereof.

The anion of the tin-containing compound is selected from the group consisting of F⁻, Cl⁻, Br⁻, I⁻, SO₄²⁻, HSO₄⁻, CH₃SO₃⁻, C₆H₅SO₃⁻, H₂PO₄⁻, HPO₄²⁻, PO₄³⁻, PF₆⁻, BO₂⁻, BF₄⁻, SiF₆²⁻, and CH₃CO₂⁻.

In a specific embodiment, the catalyst of the current invention is a combination of 1,3-dimethylimidazolium methyl sulfate and SnCl₄.5H₂O.

In another specific embodiment, the catalyst is a combination of 1-ethyl-3-methylimidazolium chloride and SnCl₄.5H₂O.

In another specific embodiment, the catalyst is a combination of 1,3-dimethylimidazolium methyl sulfate and SnCl₂.

In another specific embodiment, the catalyst is a combination of 1-ethyl-3-methylimidazolium chloride and Sn(CH₃SO₃⁻)₂.

In another specific embodiment, the catalyst is a combination of 1-ethyl-3-methylimidazolium chloride and Sn(C₆H₅SO₃⁻)₂.

According to the method of the current invention, the solvent comprises a polar solvent, such as water, or alcohols, or mixtures thereof, which could dissolve the catalyst to form a homogeneous catalyst solution.

More specifically, the alcohol is selected from the group consisting of methanol, ethanol, 1-propanol, 2-propanol, 1-butanol, 2-butanol, tert-butanol, ethylene glycol, 1,2-propandiol, 1,3-propandiol, and glycerol.

In another specific embodiment, the mixture of lactic acid and alkyl lactate is prepared by heating a mixture of carbohydrates, alcohols, solvents, and nitrogen-heterocycle aromatic ring cation salts and metal compounds as catalysts in a one-pot reactor.

The amount of alcohol is about at least one mass times or more with respect to amount of carbohydrate in the carbohydrate-containing raw material. In a specific embodiment, the ratio of alcohol to carbohydrate in the carbohydrate-containing raw material by mass is about at least 3:2.

In the current invention, the heat processing of carbohydrate-containing raw material is carried out between 25 and 200° C. In a specific embodiment, the reactants solution is allowed to carry out reaction at a temperature between 25 and 180° C. Yet more specifically, the carbohydrate-containing raw material is cellulose and the reaction temperature is between 80 and 180° C.; more preferably, the reaction temperature is between 100 and 180° C.

In a specific embodiment, the carbohydrate-containing raw material is starch and the reaction temperature is between 80 and 180° C.; more preferably, the reaction temperature is between 80 and 160° C.

In a specific embodiment, the carbohydrate-containing raw material is sucrose and the reaction temperature is between 25 and 180° C.; more preferably, the reaction temperature is between 25 and 140° C.

In a specific embodiment, the carbohydrate-containing raw material is glucose and the reaction temperature is between 25 and 180° C.; more preferably, the reaction temperature is between 25 and 140° C.

According to the current invention, a carbohydrate-containing raw material is heat-processed in a solvent in the presence of catalyst, to obtain lactic acid and/or lactate. The carbohydrate-containing raw material is processed by an environment-friendly method. Lactic acid and/or lactate is manufactured efficiently and simply by processing the carbohydrate-containing raw material under mild conditions. In addition, polylactic acid could be produced in the process as a by-product. With the method of current invention, effective usage of carbohydrate-containing raw material is enabled.

### BRIEF DESCRIPTION OF THE DRAWINGS

FIG. 1 shows the scheme for lactic acid and alkyl lactate preparation at modern industrial plants.

FIG. 2 shows one embodiment of the present invention.

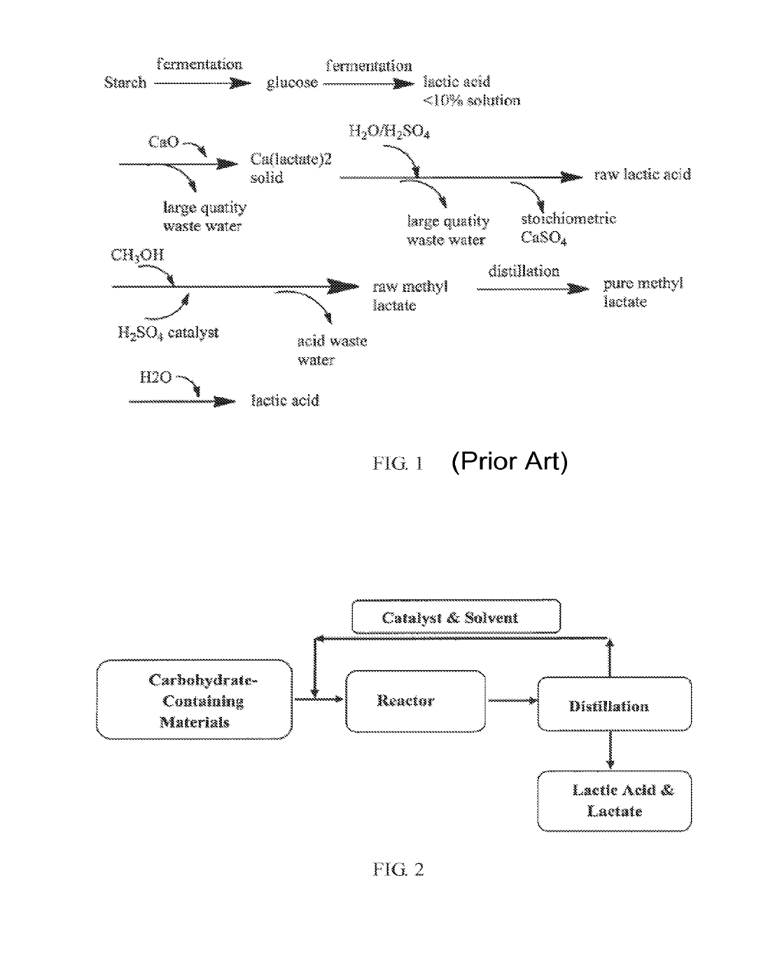

### DETAILED DESCRIPTION OF PREFERRED EMBODIMENTS

The detailed descriptions of the present invention set forth below in connection with the examples are preferred embodiments of the present invention, but the present invention is not limited to the embodiments and forms described hereinafter.

### Example 1

### Reaction Results of Fructose

The results listed in Table 1 were obtained using 1,3-dimethylimidazolium methylsulfate and SnCl₄.5H₂O as catalyst. After adding 1,3-dimethylimidazolium methylsulfate (see the amount in Table 1), SnCl₄.5H₂O (see the amount in Table 1), 0.200 g of fructose, and 5.0 mL of methanol into a 10 mL batch reactor, the reactor was sealed and heated to 140° C. under stirring to carry out the reaction. The reaction time is listed in Table 1. After reaction, NaOH solution (0.50M, 10.0 mL) was added to carry out a hydrolysis reaction at 60° C. for 5 hours to obtain a solution. HCl solution (0.50M, 10.0 mL) was added into the resulting solution to convert sodium lactate to lactic acid, and then the solution was analyzed on a HPLC to obtain the total percent yield of lactic acid and methyl lactate (as that listed in Table 1). In Table 1, “DMIMMS” stands for the 1,3-dimethylimidazolium methylsulfate; “t” stands for reaction time in hours; “T” stands for the reaction temperature in degrees Celsius; and “Y” stands for the total percent yield of lactic acid and methyl lactate.

TABLE 1
Reaction results of fructose

| SnCl₄•5H₂O | DMIMMS | | t | T | Y |
|---|---|---|---|---|---|
| (g) | (g) | Carbohydrate | (h) | (° C.) | (%) |
| 0.2 | 0.200 | fructose | 2.0 | 140 | 94 |
| 0.2 | 0.200 | fructose | 4.0 | 140 | 91 |
| 0.2 | 0 | fructose | 2.0 | 140 | 28 |

### Example 2

### Reaction Results of Glucose

The results listed in Table 2 were obtained using 1,3-dimethylimidazolium methylsulfate and SnCl₄.5H₂O as catalyst. After adding 1,3-dimethylimidazolium methylsulfate (see the amount in Table 2), SnCl₄.5H₂O (see the amount in Table 2), 0.200 g of glucose, and 5.0 mL of methanol into a 10 mL batch reactor, the reactor was sealed and heated to 140° C. under stirring to carry out the reaction. The reaction time is listed in Table 2. After reaction, NaOH solution (0.50M, 10.0 mL) was added to carry out a hydrolysis reaction at 60° C. for 5 hours to obtain a solution. HCl solution (0.50M, 10.0 mL) was added into the resulting solution to convert sodium lactate to lactic acid, and then the solution was analyzed on a HPLC to obtain the total percent yield of lactic acid and methyl lactate (as that listed in Table 2). In Table 2, “DMIMMS” stands for the 1,3-dimethylimidazolium methylsulfate; “t” stands for reaction time in hours; “T” stands for the reaction temperature in degrees Celsius; and “Y” stands for the total percent yield of lactic acid and methyl lactate.

TABLE 2
Reaction results of glucose

| SnCl₄•5H₂O | DMIMMS | | t | T | Y |
|---|---|---|---|---|---|
| (g) | (g) | Carbohydrate | (h) | (° C.) | (%) |
| 0.2 | 0 | glucose | 2 | 140 | 26 |
| 0.2 | 0.200 | glucose | 2 | 140 | 64 |
| 0.2 | 0.200 | glucose | 4 | 140 | 66 |

### Example 3

### Reaction Results of Sucrose

The results listed in Table 3 were obtained using different 1,3-dialkyl imidazolium salts and SnCl₄.5H₂O as catalyst. After adding 1,3-dialkyl imidazolium salt (see the amount in Table 3), SnCl₄.5H₂O (see the amount in Table 3), 0.200 g of sucrose, and 5.0 mL of methanol into a 10 mL batch reactor, the reactor was sealed and heated to reaction temperature (listed in Table 3) under stirring to carry out the reaction. The reaction time is listed in Table 3. After reaction, NaOH solution (0.50 M, 10.0 mL) was added to carry out a hydrolysis reaction at 60° C. for 5 hours to obtain a solution. HCl solution (0.50 M, 10.0 mL) was added into the resulted solution to convert sodium lactate to lactic acid. The solution was analyzed on a HPLC to obtain the total percent yield of lactic acid and methyl lactate (as that listed in Table 3). In Table 3, “t” stands for reaction time in hours; “T” stands for the reaction temperature in degrees Celsius; and “Y” stands for the total percent yield of lactic acid and methyl lactate. DMDIMDC has the following structure:

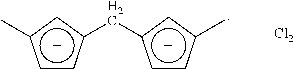

TABLE 3
Reaction results of sucrose

| SnCl₄•5H₂O | 1,3-dialkyl imidazolium | t | T | Y |
|---|---|---|---|---|
| (g) | salt (g) | (h) | (° C.) | (%) |
| 0.200 | DMIMMS (0.200) | 4 | 140 | 54 |
| 0.200 | DMIMMS (0.200) | 10 | 140 | 55 |
| 0.200 | DMIMMS (0.200) | 15 | 140 | 61 |
| 0.200 | DMIMMS (0.200) | 20 | 140 | 59 |
| 0.200 | DMIMMS (0.500) | 4 | 140 | 60 |
| 0.500 | DMIMMS (0.200) | 4 | 140 | 66 |
| 0.500 | DMIMMS (0.500) | 4 | 140 | 72 |
| 0.200 | DMIMMS (0.200) | 15 | 150 | 55 |
| 0.200 | DMIMMS (0.200) | 15 | 160 | 50 |
| 0.200 | DMIMMS (0.200) | 15 | 170 | 43 |
| 0.200 | methyl pyridine | 15 | 140 | 7 |
| | sulfate (0.200) | | | |
| 0.200 | N-methyl-N-ethyl- | 15 | 140 | 26 |
| | imidazolium chloride | | | |
| | (0.200) | | | |
| 0.200 | DMDIMDC (0.200) | 15 | 140 | 29 |

### Example 4

### Reaction Results of Starch

The results listed in Table 4 were obtained using different amounts of DMIMMS and SnCl₄.5H₂O as catalyst. After adding DMIMMS (see the amount in Table 4), SnCl₄.5H₂O (see the amount in Table 4), water (1.0 g), 0.200 g of starch, and 5.0 mL of methanol into a 10 mL batch reactor, the reactor was sealed and heated to reaction temperature (listed in Table 4) under stirring to carry out the reaction. The reaction time is listed in Table 4. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 4). In Table 4, “t” stands for reaction time in hours; “T” stands for the reaction temperature in degrees Celsius; and “Y” stands for the total percent yield of lactic acid and methyl lactate.

TABLE 4
Reaction results of starch

| H₂O | SnCl₄•5H₂O | DMIMMS | t | T | Y |
|---|---|---|---|---|---|
| (g) | (g) | (g) | (h) | (° C.) | (%) |
| 1.0 | 0.200 | 0.500 | 8 | 170 | 16 |
| 1.0 | 0.500 | 0.200 | 8 | 170 | 36 |
| 1.0 | 0.500 | 0.500 | 8 | 170 | 40 |
| 1.0 | 0.200 | 0.500 | 8 | 150 | 37 |
| 1.0 | 0.500 | 0.200 | 8 | 150 | 45 |
| 1.0 | 0.500 | 0.500 | 8 | 150 | 55 |
| 1.0 | 0.200 | 0.200 | 15 | 160 | 33 |
| 1.0 | 0.200 | 0.200 | 15 | 180 | 30 |
| 1.0 | 0.200 | 0.200 | 15 | 150 | 41 |
| 1.0 | 0.200 | 0.200 | 15 | 160 | 33 |
| 1.0 | 0.200 | 0.200 | 15 | 170 | 25 |
| 1.0 | 0.200 | 0 | 10 | 140 | 6 |
| 1.0 | 0.200 | 0.200 | 10 | 140 | 39 |
| 1.0 | 0.200 | 0.200 | 15 | 140 | 32 |
| 1.0 | 0.200 | 0.500 | 15 | 140 | 37 |
| 1.0 | 0.500 | 0.200 | 15 | 140 | 45 |
| 1.0 | 0.500 | 0.500 | 15 | 140 | 54 |

### Example 5

### Reaction Results of Cellulose

The results listed in Table 5 were obtained using DMIMMS and SnCl₄.5H₂O as catalyst. After adding DMIMMS (see the amount in Table 5), SnCl₄.5H₂O (see the amount in Table 5), water (1.0 g), 0.200 g of cellulose, and 5.0 mL of methanol into a 10 mL batch reactor, the reactor was sealed and heated to reaction temperature (listed in Table 5) under stirring to carry out the reaction. The reaction time is listed in Table 5. After reaction, NaOH solution (0.50 M, 10.0 mL) was added carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 5). In Table 5, “t” stands for reaction time in hours; “T” stands for the reaction temperature in degrees Celsius; and “Y” stands for the total percent yield of lactic acid and methyl lactate.

TABLE 5
Reaction results of cellulose

| H₂O | SnCl₄•5H₂O | DMIMMS | t | T | Y |
|---|---|---|---|---|---|
| (g) | (g) | (g) | (h) | (° C.) | (%) |
| 1.0 | 0.2 | 0.2 | 15 | 160 | 3 |
| 1.0 | 0.2 | 0.2 | 15 | 170 | 11 |
| 1.0 | 0.2 | 0.2 | 15 | 180 | 9 |

### Example 6

### Reaction Results of Corn Starch

The results listed in Table 6 were obtained using 1-ethyl-3-methylimidazolium chloride (EMIMC) and SnCl₄.5H₂O as catalyst. After adding 1-ethyl-3-methylimidazolium chloride (see the amount in Table 6), SnCl₄.5H₂O (see the amount in Table 6), water (1.0 g), 0.500 g of starch, and 4.0 mL of methanol into a 10 mL batch reactor, the reactor was sealed and heated to reaction temperature (listed in Table 6) under stirring to carry out the reaction. The reaction time is listed in Table 6. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 6). In Table 6, “t” stands for reaction time in hours; “T” stands for the reaction temperature in degrees Celsius; and “Y” stands for the total yield of lactic acid and methyl lactate.

TABLE 6
Reaction results of corn starch

| H₂O | SnCl₄•5H₂O | EMIMC | t | T | Y |
|---|---|---|---|---|---|
| (g) | (g) | (g) | (h) | (° C.) | (%) |
| 1.0 | 0.5005 | 1.0112 | 2 | 160 | 27 |
| 1.0 | 0.5004 | 1.0092 | 6 | 160 | 32 |
| 1.0 | 0.4999 | 1.0465 | 4 | 170 | 34 |
| 1.0 | 0.5005 | 1.0384 | 6 | 170 | 36 |
| 1.0 | 0.5005 | 1.0321 | 7 | 170 | 37 |
| 1.0 | 0.4998 | 1.0104 | 8 | 170 | 39 |
| 1.0 | 0.5004 | 1.0035 | 10 | 170 | 39 |
| 1.0 | 0.5002 | 1.0083 | 15 | 170 | 40 |

### Example 7

### Reaction Results of Sucrose

The results listed in Table 7 were obtained using 1,3-dimethylimidazolium sulfate ((DMIM)₂SO₄) and SnCl₄.5H₂O as catalyst. After adding (DMIM)₂SO₄ (see the amount in Table 7), SnCl₄.5H₂O (see the amount in Table 7), water (1.0 g), 0.500 g of sucrose, and methanol (5.0 mL) were added into a 10 mL batch reactor, the reactor was sealed and heated to reaction temperature (listed in Table 7) under stirring to carry out the reaction for 2 hours. The reaction time is listed in Table 7. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 7). In Table 7, “t” stands for reaction time in hours; “T” stands for the reaction temperature in degrees Celsius; and “Y” stands for the total percent yield of lactic acid and methyl lactate.

TABLE 7
Reaction results of sucrose

| H₂O | SnCl₄•5H₂O | (DMIM)₂SO₄ | t | T | Y |
|---|---|---|---|---|---|
| (g) | (g) | (g) | (h) | (° C.) | (%) |
| 1.0 | 1.00 | 1.00 | 2 | 160 | 10 |
| 1.0 | 1.00 | 1.00 | 2 | 140 | 12 |
| 1.0 | 1.00 | 1.00 | 2 | 120 | 10 |
| 1.0 | 1.00 | 1.00 | 2 | 100 | 40 |

### Example 8

### Reaction Results of Sucrose

The results listed in Table 8 were obtained using 1,3-dimethylimidazolium sulfate ((DMIM)₂SO₄) and SnCl₄.5H₂O as catalyst. After adding (DMIM)₂SO₄ (see the amount in Table 8), SnCl₄.5H₂O (see the amount in Table 8), water (1.0 g), 0.200 g of sucrose, and methanol (5.0 mL) were added into a 10 mL batch reactor, the reactor was sealed and heated to reaction temperature (listed in Table 8) under stirring to carry out the reaction for 2 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 8). In Table 8, “t” stands for reaction time in hours; “T” stands for the reaction temperature in degrees Celsius; and “Y” stands for the total yield of lactic acid and methyl lactate.

TABLE 8
Reaction results of sucrose

| H₂O | SnCl₄•5H₂O | (DMIM)₂SO₄ | t | T | Y |
|---|---|---|---|---|---|
| (g) | (g) | (g) | (h) | (° C.) | (%) |
| 1.0 | 1.00 | 1.00 | 2 | 160 | 11 |
| 1.0 | 1.00 | 1.00 | 2 | 140 | 26 |
| 1.0 | 1.00 | 1.00 | 2 | 120 | 28 |
| 1.0 | 1.00 | 1.00 | 2 | 100 | 78 |
| 1.0 | 1.00 | 1.00 | 2 | 80 | 75 |

### Example 9

### Reaction Results of Glucose

The results listed in Table 9 were obtained using 1,3-dimethylimidazolium sulfate ((DMIM)₂SO₄) and SnCl₄.5H₂O as catalyst. After adding (DMIM)₂SO₄ (see the amount in Table 9), SnCl₄.5H₂O (see the amount in Table 9), water (1.0 g), 0.500 g of glucose, and methanol (5.0 mL) were added into a 10 mL batch reactor, the reactor was sealed and heated to reaction temperature (listed in Table 9) under stirring to carry out the reaction for 5 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 9). In Table 9, “t” stands for reaction time in hours; “T” stands for the reaction temperature in degrees Celsius; and “Y” stands for the total percent yield of lactic acid and methyl lactate.

TABLE 9
Reaction results of glucose

| H₂O | SnCl₄•5H₂O | (DMIM)₂SO₄ | t | T | Y |
|---|---|---|---|---|---|
| (g) | (g) | (g) | (h) | (° C.) | (%) |
| 1.0 | 1.00 | 1.00 | 5 | 160 | 14 |
| 1.0 | 1.00 | 1.00 | 5 | 140 | 16 |
| 1.0 | 1.00 | 1.00 | 5 | 120 | 25 |
| 1.0 | 1.00 | 1.00 | 5 | 100 | 34 |

### Example 10

### Reaction Results of Glucose

The results listed in Table 10 were obtained using 1,3-dimethylimidazolium sulfate ((DMIM)₂SO₄) and SnCl₄.5H₂O as catalyst. After adding (DMIM)₂SO₄ (see the amount in Table 10), SnCl₄.5H₂O (see the amount in Table 10), water (1.0 g), 0.200 g of glucose, and methanol (5.0 mL) were added into a 10 mL batch reactor, the reactor was sealed and heated to reaction temperature (listed in Table 10) under stirring to carry out the reaction for 2 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 10). In Table 10, “t” stands for reaction time in hours; “T” stands for the reaction temperature in degrees Celsius; and “Y” stands for the total percent yield of lactic acid and methyl lactate.

TABLE 10
Reaction results of glucose

| H₂O | SnCl₄•5H₂O | (DMIM)₂SO₄ | t | T | Y |
|---|---|---|---|---|---|
| (g) | (g) | (g) | (h) | (° C.) | (%) |
| 1.0 | 1.00 | 1.00 | 5 | 140 | 24 |
| 1.0 | 1.00 | 1.00 | 5 | 120 | 31 |
| 1.0 | 1.00 | 1.00 | 5 | 100 | 75 |

### Example 11

### Reaction Results of Starch

The results listed in Table 11 were obtained using 1,3-dimethylimidazolium sulfate ((DMIM)₂SO₄) and SnCl₄.5H₂O as catalyst. After adding (DMIM)₂SO₄ (see the amount in Table 11), SnCl₄.5H₂O (see the amount in Table 11), water (1.0 g), starch, and methanol (5.0 mL) were added into a 10 mL batch reactor, the reactor was sealed and heated to reaction temperature (listed in Table 11) under stirring to carry out the reaction for 5 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 11). In Table 11, “T” stands for the reaction temperature in degrees Celsius and “Y” stands for the total percent yield of lactic acid and methyl lactate.

TABLE 11
Reaction results of starch

| H₂O | SnCl₄•5H₂O | (DMIM)₂SO₄ | starch | T | Y |
|---|---|---|---|---|---|
| (g) | (g) | (g) | (g) | (° C.) | (%) |
| 1.0 | 1.00 | 1.00 | 0.500 | 140 | 22 |
| 1.0 | 1.00 | 1.00 | 0.500 | 120 | 10 |
| 1.0 | 1.00 | 1.00 | 0.200 | 160 | 30 |
| 1.0 | 1.00 | 1.00 | 0.200 | 100 | 8 |

### Example 12

### Reaction Results of Sucrose

The results listed in Table 12 were obtained using 1,3-dimethylimidazolium hydrogen sulfate (DMIMHSO₄) and SnCl₄.5H₂O as catalyst. After adding DMIMHSO₄ (see the amount in Table 12), SnCl₄.5H₂O (see the amount in Table 12), sucrose, and methanol (5.0 mL) were added into a 10 mL batch reactor, the reactor was sealed and heated to reaction temperature (listed in Table 12) under stirring to carry out the reaction for 5 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 12). In Table 12, “T” stands for the reaction temperature in degrees Celsius and “Y” stands for the total percent yield of lactic acid and methyl lactate.

TABLE 12
Reaction results of sucrose

| SnCl₄•5H₂O | DMIMHSO₄ | sucrose | T | Y |
|---|---|---|---|---|
| (g) | (g) | (g) | (° C.) | (%) |
| 1.00 | 1.00 | 0.500 | 160 | 10 |
| 1.00 | 1.00 | 0.500 | 140 | 18 |
| 1.00 | 1.00 | 0.500 | 120 | 16 |

### Example 13

### Reaction Results of Sucrose

The results listed in Table 13 were obtained using 1,3-dimethylimidazolium hydrogen sulfate (DMIMHSO₄) and SnCl₄.5H₂O as catalyst. After adding DMIMHSO₄ (see the amount in Table 13), SnCl₄.5H₂O (see the amount in Table 13), glucose, and methanol (5.0 mL) were added into a 10 mL batch reactor, the reactor was sealed and heated to reaction temperature (listed in Table 13) under stirring to carry out the reaction for 5 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 13). In Table 13, “T” stands for the reaction temperature in degrees Celsius and “Y” stands for the total percent yield of lactic acid and methyl lactate.

TABLE 13
Reaction results of glucose

| SnCl₄•5H₂O | DMIMHSO₄ | glucose | T | Y |
|---|---|---|---|---|
| (g) | (g) | (g) | (° C.) | (%) |
| 1.00 | 1.00 | 0.500 | 160 | 23 |
| 1.00 | 1.00 | 0.500 | 140 | 8 |
| 1.00 | 1.00 | 0.500 | 120 | 9 |

### Example 14

### Reaction Results of Starch

The results listed in Table 14 were obtained using 1,3-dimethylimidazolium hydrogen sulfate (DMIMHSO₄) and SnCl₄.5H₂O as catalyst. After adding DMIMHSO₄ (see the amount in Table 14), SnCl₄.5H₂O (see the amount in Table 14), starch, and 5.0 mL of methanol added into a 10 mL batch reactor, the reactor was sealed and heated to reaction temperature (listed in Table 14) under stirring to carry out the reaction for 5 hours. After reaction, NaOH solution (0.50 M, 10.0 mL) was added to carry out a hydrolysis reaction at 60° C. for 5 hours to obtain a solution. HCl (0.50 M, 10.0 mL) was added into the resulting solution to convert sodium lactate to lactic acid, and then the solution was analyzed on a HPLC to obtain the total percent yield of lactic acid and methyl lactate (as that listed in Table 14). In Table 14, “T” stands for the reaction temperature in degrees Celsius and “Y” stands for the total yield of lactic acid and methyl lactate.

TABLE 14
Reaction results of starch

| | CH₃OH | SnCl₄•5H₂O | DMIMHSO₄ | starch | | |
|---|---|---|---|---|---|---|
| H₂O (g) | (mL) | (g) | (g) | (g) | T (° C.) | Y (%) |
| 0 | 5.0 | 1.00 | 1.00 | 0.500 | 160 | 20 |
| 1.0 | 4.0 | 1.00 | 1.00 | 0.500 | 160 | 22 |
| 0 | 5.0 | 1.00 | 1.00 | 0.200 | 80 | 2 |

### Example 15

The results listed in Table 15 were obtained using 1-ethyl-3-methylimidazolium chloride (EMIMC) and Sn(CH₃SO₃)₂ as catalyst. After adding EMIMC (see the amount in Table 15), Sn(CH₃SO₃)₂, sucrose, and methanol were added into a batch reactor (volume 15 mL), the reactor was sealed and heated to reaction temperature (listed in Table 15) under stifling to carry out the reaction for 2 hours. After reaction, the solution was analyzed on a GC to obtain the total percent yield of methyl lactate (as that listed in Table 15). In Table 15, “T” stands for the reaction temperature in degrees Celsius and “Yₘₗ” stands for the total yield of methyl lactate.

TABLE 15
The reaction results of sucrose at different temperature

| T | Sn(CH₃SO₃)₂ | sucrose | CH₃OH | EMIMC | Yₘₗ |
|---|---|---|---|---|---|
| (° C.) | (g) | (g) | (mL) | (g) | (%) |
| 80 | 0.20 | 0.20 | 8.0 | 0.50 | 1 |
| 90 | 0.20 | 0.20 | 8.0 | 0.50 | 20 |
| 100 | 0.20 | 0.20 | 8.0 | 0.50 | 41 |
| 110 | 0.20 | 0.20 | 8.0 | 0.50 | 43 |
| 120 | 0.20 | 0.20 | 8.0 | 0.50 | 42 |
| 130 | 0.20 | 0.20 | 8.0 | 0.50 | 50 |
| 140 | 0.20 | 0.20 | 8.0 | 0.50 | 41 |

### Example 16

The results listed in Table 16 were obtained using 1-ethyl-3-methylimidazolium chloride (EMIMC) and Sn(CH₃SO₃)₂ as catalyst. After adding EMIMC (see the amount in Table 16), Sn(CH₃SO₃)₂, sucrose, and methanol were added into a batch reactor (volume 15 mL), the reactor was sealed and heated to 130° C. under stirring to carry out the reaction from 0.5 to 4 hours. After reaction, the solution was analyzed on a GC to obtain the total percent yield of methyl lactate (as that listed in Table 16). In Table 16, “t” stands for the reaction time in hours and “Yₘₗ” stands for the total yield of methyl lactate.

TABLE 16
The reaction results of sucrose at
130° C. for different reaction time

| t | Sn(CH₃SO₃)₂ | sucrose | CH₃OH | EMIMC | Yₘₗ |
|---|---|---|---|---|---|
| (h) | (g) | (g) | (mL) | (g) | (%) |
| 0.5 | 0.20 | 0.20 | 8.0 | 0.50 | 10 |
| 1 | 0.20 | 0.20 | 8.0 | 0.50 | 30 |
| 1.5 | 0.20 | 0.20 | 8.0 | 0.50 | 35 |
| 2 | 0.20 | 0.20 | 8.0 | 0.50 | 50 |
| 2.5 | 0.20 | 0.20 | 8.0 | 0.50 | 35 |
| 3 | 0.20 | 0.20 | 8.0 | 0.50 | 35 |
| 4 | 0.20 | 0.20 | 8.0 | 0.50 | 18 |

### Example 17

The results listed in Table 17 were obtained using different ionic liquid and Sn(CH₃SO₃)₂ as catalyst. After adding ionic liquid (0.50 g, see Table 17), Sn(CH₃SO₃)₂ (0.20 g), sucrose (0.20 g), and methanol (8.0 mL) were added into a batch reactor (volume 15 mL), the reactor was sealed and heated to 130° C. under stirring to carry out the reaction for 2 hours. After reaction, the solution was analyzed on a GC to obtain the total percent yield of methyl lactate (as that listed in Table 17). In Table 17, “Yₘₗ” stands for the total yield of methyl lactate.

### Note:

DMDIMDC stands for the following compound:

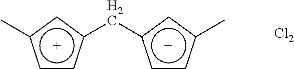

DMBIMC stands for the following compound:

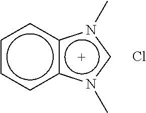

TABLE 17
The reaction results of by using different ionic liquids

| | Yₘₗ |
|---|---|
| ionic liquid | (%) |
| 1-ethyl-3-methylimidazolium chloride | 50 |
| DMDTMDC | 65 |
| 1-butyl-2,3-dimethylimidazolium chloride | 59 |
| DMBIMC | 13 |
| 1,3-dimethylimidazolium iodide | 23 |

### Example 18

The results listed in Table 18 were obtained using 1-ethyl-3-methylimidazolium chloride (EMIMC) and Sn(CH₃SO₃)₂ as catalyst. After adding EMIMC (see the amount in Table 18), Sn(CH₃SO₃)₂, starch, and methanol were added into a batch reactor (volume 15 mL), the reactor was sealed and heated to 160° C. under stirring to carry out the reaction from 2 to 15 hours. After reaction, the solution was analyzed on a GC to obtain the total percent yield of methyl lactate (as that listed in Table 18). In Table 18, “t” stands for the reaction time in hours and “Yₘₗ” stands for the total yield of methyl lactate.

TABLE 18
The reaction results of starch at 160° C. for different reaction time

| t | Sn(CH₃SO₃)₂ | starch | CH₃OH | EMIMC | Yₘₗ |
|---|---|---|---|---|---|
| (h) | (g) | (g) | (mL) | (g) | (%) |
| 2 | 0.20 | 0.20 | 8.0 | 0.50 | 3 |
| 4 | 0.20 | 0.20 | 8.0 | 0.50 | 11 |
| 6 | 0.20 | 0.20 | 8.0 | 0.50 | 16 |
| 8 | 0.20 | 0.20 | 8.0 | 0.50 | 32 |
| 10 | 0.20 | 0.20 | 8.0 | 0.50 | 32 |
| 12 | 0.20 | 0.20 | 8.0 | 0.50 | 36 |
| 15 | 0.20 | 0.20 | 8.0 | 0.50 | 31 |

### Example 19

The results listed in Table 19 were obtained using 1-ethyl-3-methylimidazolium chloride (EMIMC) and Sn(CH₃SO₃)₂ as catalyst. After adding EMIMC (see the amount in Table 19), Sn(CH₃SO₃)₂, starch, and methanol were added into a batch reactor (volume 15 mL), the reactor was sealed and heated to reaction temperature (listed in Table 19) under stirring to carry out the reaction for 8 hours. After reaction, the solution was analyzed on a GC to obtain the total percent yield of methyl lactate (as that listed in Table 19). In Table 19, “T” stands for the reaction temperature in degrees Celsius and “Yₘₗ” stands for the total yield of methyl lactate.

TABLE 19
The reaction results of starch at different temperature

| T | Sn(CH₃SO₃)₂ | starch | CH₃OH | EMIMC | Yₘₗ |
|---|---|---|---|---|---|
| (° C.) | (g) | (g) | (mL) | (g) | (%) |
| 150 | 0.20 | 0.20 | 8.0 | 0.50 | 1 |
| 160 | 0.20 | 0.20 | 8.0 | 0.50 | 20 |
| 170 | 0.20 | 0.20 | 8.0 | 0.50 | 41 |
| 180 | 0.20 | 0.20 | 8.0 | 0.50 | 43 |
| 190 | 0.20 | 0.20 | 8.0 | 0.50 | 42 |

### Example 20

The results listed in Table 20 were obtained using DMDIMDBS (see structure below) and Sn(C₆H₅SO₃)₂ as catalyst. After adding DMDIMDBS (see the amount in Table 20), Sn(C₆H₅SO₃)₂, sweet potato, and methanol were added into a batch reactor (volume 15 mL), the reactor was sealed and heated to reaction temperature (listed in Table 20) under stirring to carry out the reaction for 8 hours. After reaction, the solution was analyzed on a GC to obtain the total percent yield of methyl lactate (as that listed in Table 20). In Table 20, “T” stands for the reaction temperature in degrees Celsius and “Yₘₗ” stands for the total yield of methyl lactate. DMDIMDBS stands for the following compound:

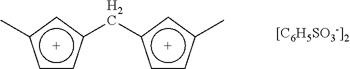

TABLE 20
The reaction results of sweet potato
(dry powder) at different temperature

| | | Sweet | | | |
|---|---|---|---|---|---|
| T | Sn(C₆H₅SO₃)₂ | potato | CH₃OH/H₂O | DMDIMDBS | Yₘₗ |
| (° C.) | (g) | (g) | (g/g) | (g) | (%) |
| 160 | 0.30 | 0.20 | 6.4/0.30 | 0.50 | 34 |
| 160 | 0.30 | 0.20 | 4.8/0.30 | 0.50 | 37 |
| 160 | 0.30 | 0.20 | 3.2/0.30 | 0.50 | 28 |
| 160 | 0.30 | 0.20 | 1.6/0.30 | 0.50 | 21 |
| 140 | 0.30 | 0.20 | 6.4/0.30 | 0.50 | 17 |
| 150 | 0.30 | 0.20 | 6.4/0.30 | 0.50 | 23 |
| 160 | 0.30 | 0.20 | 6.4/0.30 | 0.50 | 34 |
| 170 | 0.30 | 0.20 | 6.4/0.30 | 0.50 | 38 |
| 180 | 0.30 | 0.20 | 6.4/0.30 | 0.50 | 36 |

### Example 21

The results listed in Table 21 were obtained using DMDIMDBS (see structure below) and Sn(C₆H₅SO₃)₂ as catalyst. After adding DMDIMDBS (see the amount in Table 21), Sn(C₆H₅SO₃)₂, sucrose, and methanol were added into a batch reactor (volume 15 mL), the reactor was scaled and heated to reaction temperature (listed in Table 21) under stirring to carry out the reaction for 2 hours. After reaction, the solution was analyzed on a GC to obtain the total percent yield of methyl lactate (as that listed in Table 21). In Table 21, “T” stands for the reaction temperature in degrees Celsius and “Yₘₗ” stands for the total yield of methyl lactate.

DMDIMDBS stands for the following compound:

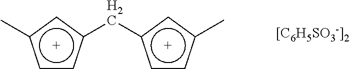

TABLE 21
The reaction results of sucrose at different temperature

| T | Sn(C₆H₅SO₃)₂ | sucrose | CH₃OH/H₂O | DMDIMDBS | Yₘₗ |
|---|---|---|---|---|---|
| (° C.) | (g) | (g) | (g/g) | (g) | (%) |
| 150 | 0.30 | 0.20 | 6.4/0 | 0.50 | 33 |
| 130 | 0.30 | 0.20 | 6.4/0 | 0.50 | 25 |
| 130 | 0.30 | 0.20 | 4.8/0 | 0.50 | 28 |
| 130 | 0.30 | 0.20 | 4.8/0.20 | 0.50 | 34 |
| 120 | 0.30 | 0.20 | 4.8/0.20 | 0.50 | 27 |
| 130 | 0.30 | 0.20 | 4.8/0.20 | 0.50 | 34 |
| 140 | 0.30 | 0.20 | 4.8/0.20 | 0.50 | 34 |
| 150 | 0.30 | 0.20 | 4.8/0.20 | 0.50 | 39 |
| 160 | 0.30 | 0.20 | 4.8/0.20 | 0.50 | 42 |

### Example 22

The results listed in Table 22 were obtained using DMDIMDBS (see structure below) and Sn(C₆H₅SO₃)₂ as catalyst. After adding DMDIMDBS (see the amount in Table 22), Sn(C₆H₅SO₃)₂, starch, and methanol were added into a batch reactor (volume 15 mL), the reactor was sealed and heated to reaction temperature (listed in Table 22) under stirring to carry out the reaction for 8 hours. After reaction, the solution was analyzed on a GC to obtain the total percent yield of methyl lactate (as that listed in Table 22). In Table 22, “T” stands for the reaction temperature in degrees Celsius and “Yₘₗ” stands for the total yield of methyl lactate.

DMDIMDBS stands for the following compound:

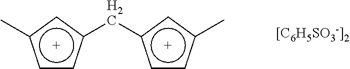

TABLE 22
The reaction results of starch at different temperature

| T | Sn(C₆H₅SO₃)₂ | Starch | CH₃OH | DMDIMDBS | Yₘₗ |
|---|---|---|---|---|---|
| (° C.) | (g) | (g) | (mL) | (g) | (%) |
| 140 | 0.282 | 0.20 | 8.0 | 0.50 | 36 |
| 150 | 0.282 | 0.20 | 8.0 | 0.50 | 32 |
| 160 | 0.282 | 0.20 | 8.0 | 0.50 | 48 |
| 170 | 0.282 | 0.20 | 8.0 | 0.50 | 42 |
| 180 | 0.282 | 0.20 | 8.0 | 0.50 | 35 |
| 160 | 0.282 | 0.20 | 8.0 | 0.50 | 37 |
| 160 | 0.282 | 0.20 | 8.0 | 0.50 | 33 |
| 160 | 0.282 | 0.20 | 8.0 | 0.50 | 42 |
| 160 | 0.282 | 0.20 | 8.0 | 0.50 | 44 |
| 160 | 0.282 | 0.20 | 8.0 | 0.50 | 48 |

We claim:

1. A method for synthesizing lactic acid and alkyl lactate from carbohydrate-containing raw materials, comprising:
(a) preparing a mixture of at least one carbohydrate-containing raw material, at least one alcohol, at least one catalyst comprising nitrogen-heterocycle aromatic cation salts and metal compounds, and at least one solvent; and
(b) heating the mixture to obtain lactic acid and alkyl lactate.

2. The method of claim 1, wherein the alkyl lactate is selected from the group consisting of methyl lactate and ethyl lactate.

3. The method of claim 1, wherein the carbohydrate is selected from the group consisting of polysaccharides and monosaccharides.

4. The method of claim 1, wherein the carbohydrate is selected from the group consisting of cotton, cellulose, starch, dextran, sucrose, fructose and glucose.

5. The method of claim 1, wherein the alcohol is selected from the group consisting of monohydroxyl alcohols, dihydroxyl alcohols, and multihydroxyl alcohols.

6. The method of claim 5, wherein the monohydroxyl alcohol is selected from the group consisting of methanol, ethanol, 1-propanol, 2-propanol, 1-butanol, 2-butanol, and tert-butanol.

7. The method of claim 5, wherein the dihydroxyl alcohol is selected from the group consisting of ethylene glycol, 1,2-propandiol, and 1,3-propandiol.

8. The method of claim 5, wherein the multihydroxyl alcohol is glycerol.

9. The method of claim 1, wherein the anion of the nitrogen-heterocycle aromatic cation salts is selected from the group consisting of F⁻, Cl⁻, Br⁻, I⁻, SO₄²⁻, CH₃SO₄⁻, CH₃SO₃⁻, C₆H₅SO₃⁻ (benzenesulfenate anion), HSO₄⁻, H₂PO₄⁻, HPO₄²⁻, PO₄³⁻, PF₆⁻, BO₂⁻, BF₄⁻, SiF₆²⁻, and CH₃CO₂⁻.

10. The method of claim 1, wherein the cation of the nitrogen-heterocycle aromatic cation salts is an organic cation that contains at least one hex-member aromatic ring and/or at least one pent-member aromatic ring that bring at least one of nitrogen atoms on the ring.

11. The method of claim 10, wherein the organic cation is selected from the group consisting of 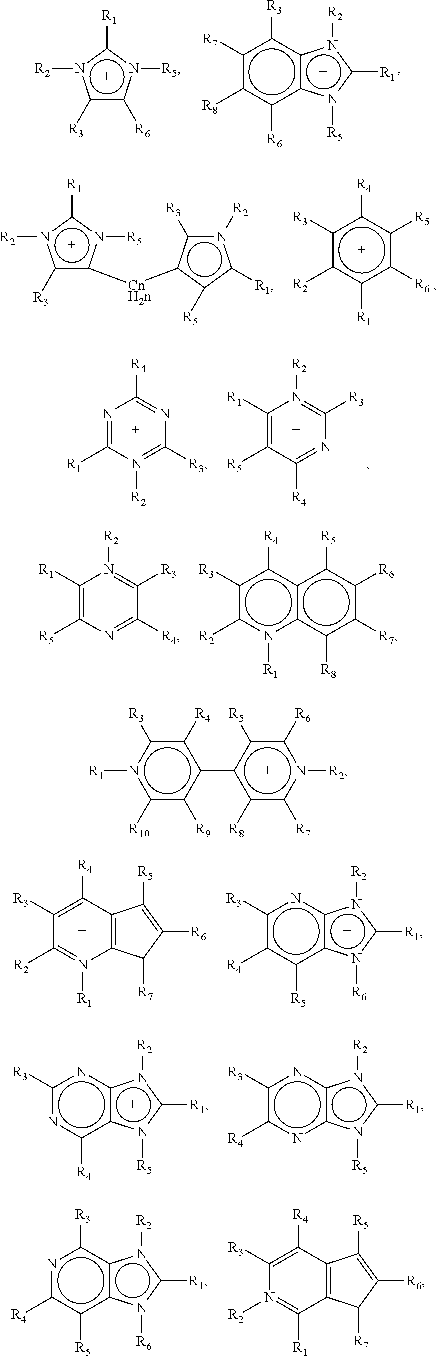 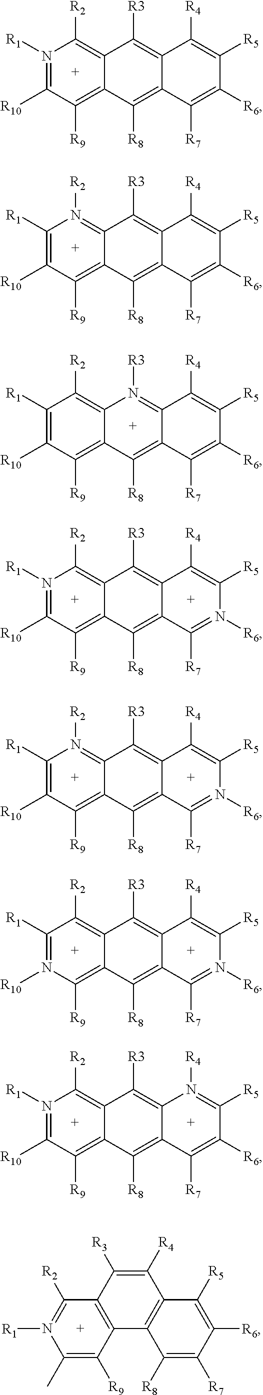 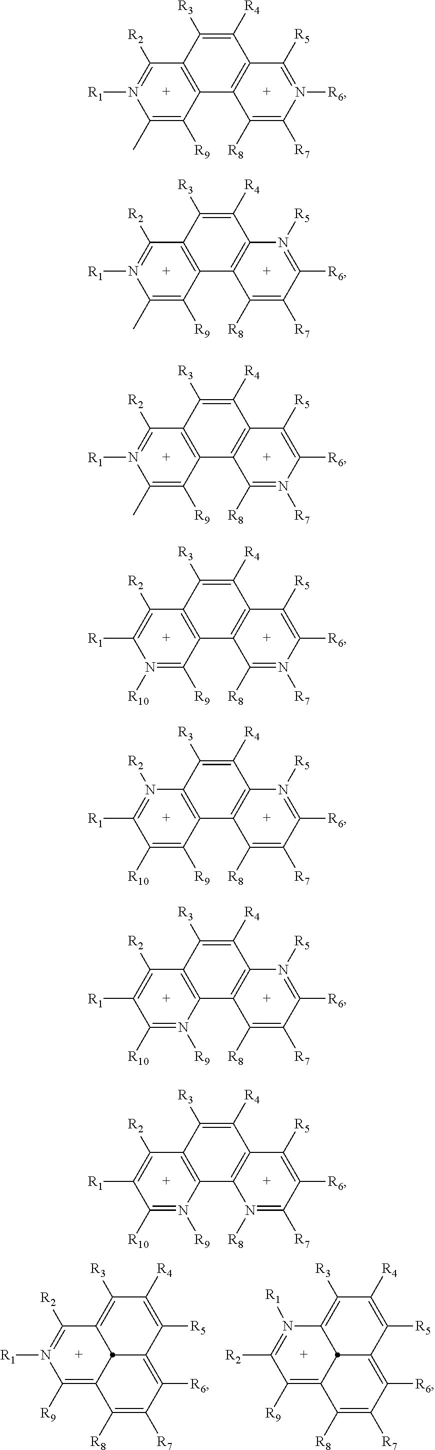 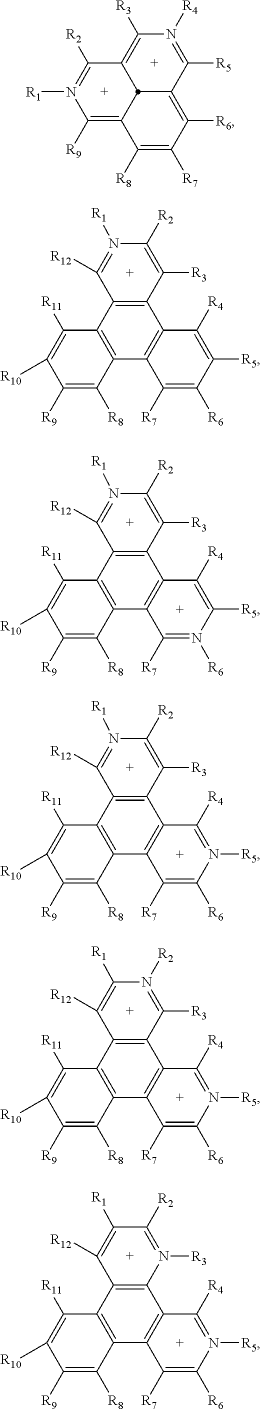 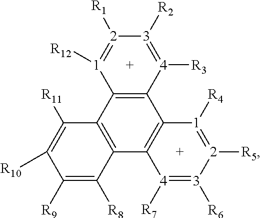
(wherein the two nitrogen atoms are respectively located on the two hex-member rings, each N atom is located at any position among 1, 2, 3, and 4 for each ring), 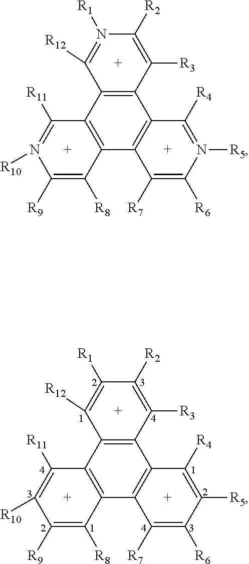
(wherein the three nitrogen atoms are respectively located on the three hex-member rings, each N atom is located at any position among 1, 2, 3 and 4 for each ring), 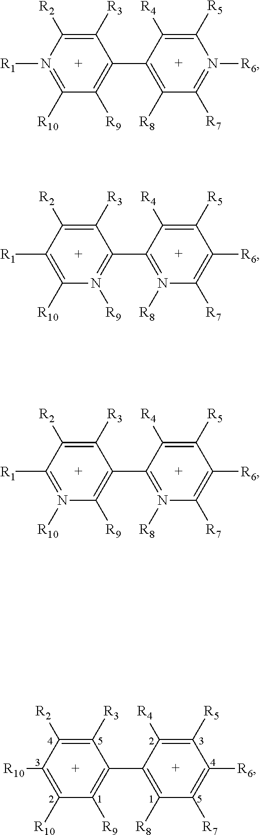
(wherein the two nitrogen atoms are respectively located on the two hex-member rings, each N atom is located at any position among 1, 2, 3 and 4 for each ring), 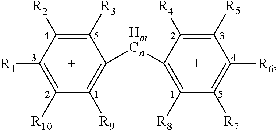
(wherein the two nitrogen atoms are respectively located on the two hex-member rings, each N atom is locate at any position among 1, 2, 3 and 4 for each ring; n and m are positive integers), and derivatives thereof, wherein the substituting group Rₙ on carbon atoms is selected from the group consisting of H—, CₙH₂ₙ₊₁— (n≧1), CₙH₂ₙ₋₁—, CₙH₂ₙ₋₃—, CₙHₘ— (m≧3), CₙH₂ₙ₋₇— (n≧6), Cl—, Br—, I—, and —OSO₃⁻.

12. The method of claim 11, wherein the substituting group Rₙ on nitrogen atoms is selected from the group consisting of CₙH₂ₙ₊₁— (n≧1), CₙH₂ₙ₋₁—, CₙH₂ₙ₋₃—, CₙHₘ— (m≧3), and CₙH₂ₙ₋₇— (n≧6).

13. The method of claim 10, wherein the organic cation is selected from the group consisting of 1,3-dimethylimidazolium, 1-ethyl-3-methylimidazolium ([EMIM]⁺), and 1,3-dimethylimidazolium ([DMIM]⁺).

14. The method of claim 1, wherein the metal compound is a tin-containing compound.

15. The method of claim 14, wherein the tin-containing compound comprises Sn⁴⁺, Sn²⁺, or mixtures thereof.

16. The method of claim 14, wherein the anion of the tin-containing compound is selected from the group consisting of F⁻, Cl⁻, Br⁻, I⁻, SO₄²⁻, HSO₄⁻, CH₃SO₃⁻, C₆H₅SO₃⁻, H₂PO₄⁻, HPO₄²⁻, PO₄³⁻, PF₆⁻, BO₂⁻, BF₄⁻, SiF₆²⁻, and CH₃CO₂⁻.

17. The method of claim 1, wherein the catalyst is a combination of 1,3-dimethylimidazolium methyl sulfate and SnCl₄.5H₂O.

18. The method of claim 1, wherein the catalyst is a combination of 1,3-dimethylimidazolium methyl sulfate and SnCl₂.

19. The method of claim 1, wherein the catalyst is a combination of 1-ethyl-3-methylimidazolium chloride and SnCl₄.5H₂O.

20. The method of claim 1, wherein the catalyst is a combination of 1-ethyl-3-methylimidazolium chloride and Sn(C₆H₅SO₃)₂.

21. The method of claim 1, wherein the catalyst is a combination of 1-ethyl-3-methylimidazolium chloride and Sn(CH₃SO₃)₂.

22. The method of claim 1, wherein the solvent is capable of dissolving the catalyst.

23. The method of claim 1, wherein the solvent comprises a polar solvent selected from the group consisting of water and alcohol.

24. The method of claim 1, wherein the alcohol is selected from the group consisting of methanol, ethanol, 1-propanol, 2-propanol, 1-butanol, 2-butanol, tert-butanol, ethylene glycol, 1,2-propandiol, 1,3-propandiol, and glycerol.

25. The method of claim 1, wherein the heating is carried out in a one-pot reactor.

26. The method of claim 1, wherein the mixture is heated up to a temperature between 25 and 200° C.

27. The method of claim 26, wherein the temperature is between 80 and 180° C.

28. The method of claim 26, wherein the temperature is between 100 and 160° C.
